(19) United States
(12) Patent Application Publication
(10) Pub. No.: US 2003/0157185 A1
(43) Pub. Date: Aug. 21, 2003

(54) Topical treatment of neuropathy

(76) Inventor: Lou Paradise, Rhinebeck, NY (US)

Correspondence Address: JOHN LEZDEY, JOHN LEZDEY AND ASSOCIATES, 4625 EAST BAY DRIVE, CLEARWATER, FL, 33764, US

(21) Appl. No.: 10/072,410

(22) Filed: Feb. 8, 2002

(51) Int. Cl. A61K 35/78; A61K 35/12; A61K 31/198

(52) U.S. Cl. 424/520; 424/737; 424/756; 424/764; 424/769; 514/565

(57) ABSTRACT

There is provided a method for treating diseases such as fibromyalgia and diabetic neuropathy by the topical application of a composition comprising plant extracts. The composition contains a lymphatic activity or and a mobilizer of white blood cell activity and a vasodilator.

### FIELD OF THE INVENTION

The present invention relates to the treatment of the symptoms of diseases relating to the restricted blood flow to nerve endings and the lower extremities of patients. More particularly, there is provided the treatment of the symptoms of such diseases as fibromyalgia, toxic neuropathy, and diabetic neuropathy.

### BACKGROUND OF THE INVENTION

Fibromyalgia is a disease characterized by pain in the lower extremities and in some cases an autoimmune disease wherein blood circulation is restricted and the nerve endings are affected.

Diabetic neuropathy occupies an important place as one of three major complications of diabetes along with retinopathy and nephropathy.

Diabetic neuropathy is currently categorized into three groups comprising mononeuropathy, symmetrical peripheral polyneuropathy and autonomic neuropathy (Williams Text Book of Endocrinology, 8^{th} Edition, p 1301, Harrison's Principles of Internal Medicine, 12^{th} edition, p 1754).

Mononeuropathy is a focal or multifocal mononeural disorder which appears as lesions of the cerebral nerve or affects soma and/or extremities. Its major symptom emerges as dyskinesia in many cases. It is known to develop more often in elder patients.

Symmetrical peripheral polyneuropathy is the most frequent form of diabetic neuropathy. It generally makes slow progress, so that its patients tend to become aware of the symptoms only after it has reached an advanced stage. The initial symptoms are often a reduced Achilles reflex parasthesis and a decline or absence of vibratory sensibility. Urtication represented as a smarting feeling and then numbness on both feet follow.

With autonomic neuropathy, patients show representative symptoms for autonomic disorder, such as orthostatic hypotension, cardiac rate alteration, dyshidrosis, atony of esophagus or gastric atony, diabetic diarrhea, impotence and others.

As mechanisms for development of these symptoms, metabolic hypothesis and vascular/ischemic hypothesis have been implied. For the former hypothesis, hypergasia of polyol metabolic pathway, a pathway where sorbitol and fructose are produced from glucose provided due to hyperglycemia is considered to be a major contributing factor. Another theory involving a reduced content of myoinositol is related to peripheral nerve disorder. In the latter hypothesis, neuro-microvascular occlusion and/or destruction of blood-nerve barrier are thought to be related to the nerve disorders.

As methods of treatment of diabetic neuropathy, it has been reported that some trial treatments have been conducted during the 1970s and 1980s based upon the hypothesis that abnormality of metabolic factors is viewed as a cause, Greene D A, DeJesus P V Jr., et al. (Effects of insulin and dietary myoinositol on impaired peripheral motor nerve conduction velocity in acute streptozatocin diabetes, J. Clin. Invest., 1975, 55, 6, 1326-36) and Yagihashi S., Nishibira M., et al (Morphometric analysis of the peripheral nerve lesions in experimental diabetes rats, Tohoku J. Exp. Med., 129, 2, 139-49, 1979). These confirmed that peripheral nerve fibers of model rats for diabetic neuropathy were morphologically impaired and NCV was reduced. In addition to that, they reported that when insuln was administered to the rats, improvements in NCV could be observed, thus finding that control over blood glucose level led to improvements in NCV.

Accordingly, current methods of treatment for diabetic neuropathy, dietary therapy and administration of insulin, both mainly proposing to control blood glucose level, administration of aldose reductase inhibitors and aminoguadanine, both mainly proposing to improve abnormal glucose metabolism, administration of troglitazone, and administration of agents for limb ischemia mainly proposing to inprove blood flow, have been conducted.

In any treatments, improvement of nerve conduction velocity was not always sufficient when a single drug was used, and methods of treatment by combined use of different therapeutic agents which have different functions have yet to be established. Accordingly, combined drug therapies for diabetic neuropathy aiming at recovering once reduced nerve conduction velocity, have not yet been confirmed.

Toxic neuropathy is similar in symptoms as diabetic neuropathy in that nerve fibers are affected and there is a resultant leg numbness or pricking in the feet.

U.S. Pat. No. 5,981,594 to Okanoto et al discloses the use of a prostaglandin I derivative together with an anti-diabetic agent to hypofunction motor and sensory nerves. However, such treatment has side effects because the prostaglandins are not localized at the sites of the pain.

Because diabetes is a systematic disease affecting many parts of the body, ideal case management requires a team approach. This is especially true for the legs, feet and digits of the feet.

U.S. Pat. No. 5,795,573 to Paradise discloses homeopathic topical pain relieving compositions for relieving pain due to injury or stress utilizing the combination of Arnica Montana, Rhus toxicodendron and Aesculus hippocastanum

### SUMMARY OF THE INVENTION

According to the invention there is provided a method of treating the symptoms of diseases related to restricted blood flow and nerve impairment wherein there is a reduction in motor and sensory nerve conduction relocation by the topical application of a composition containing vasodilators, stimulants of the lymphatic systems and mobilizers of white blood cell activity. The treatment is particularly useful for patients suffering from fibromyalgia, diabetic neuropathy, and toxic neuropathy.

The compositions of the invention include homeopathic preparations and combinations of homeopathic medicines and herbal and/or nutritional supplements.

It is advantageous to include in the compositions components which can heal the skin in those areas which have been damaged as a result of the lack of blood circulation to capillaries and blood vessels.

It is preferable that the compositions contain an effective amount of Arnica Montana to improve blood flow, Lachesis and Crotalus horridum to thin the blood and increase enzyme activity, and Echinacea to stimulate the lymphatic system to reduce inflammation.

It is a general object of the invention to provide a method for treating the pain of patients suffering from restricted blood circulation and the reduction of nerve conduction velocities.

It is a further object of the invention to treat a patient suffering from fibromyalgia or diabetic neuropathy.

It is another object of the invention to improve blood flow and the delivery of vital oxygen and nutrients to nerve cells and surrounding tissues of patients suffering from a disease which restricts blood flow to those areas which are inflamed.

These and other objects and advantages will become more apparent from a reading of the preferred embodiments of the invention.

It is understood that the term “plant” refers to flowers, herbs, vegetable extracts, tree-bark and the like.

### PREFERRED EMBODIMENTS OF THE INVENTION

According to the present invention there is provided a method of topically treating a patient suffering from a disease which causes restricted blood flow and nerve impairment, especially to the extremities. The topically applied composition can be in the form of a lotion, cream, gel or salve. It has been found that a synergistic therapeutically effective amount of the combination of a vasodilator, a stimulator of lymphatic activity and a mobilizer of white blood cell activity that can be topically applied at the site of pain and numbness is effective to provide relief from the symptom of the disease causing the problems. That is, for diseases such as fibromyalgia, diabetic neuropathy and toxic neuropathy.

More specifically, the components of the composition comprise the following:

A. Vasodilator—Arnica Montana, primrose oil, rosemary, ginger, Hamamelis, horse chestnuts, Yohimbe, niacin L-ornithate, and the like.

B. Stimulator of lymphatic activity—Echinacea Augustofolia, St. Johns Wort, Belladonna, Devils Claw, Yellow Dock, Burdock, North American Ginseng, Wild Indigo, Pleurisy Root, and Pokeweed.

C. Mobilizers of white blood cell activity—Lachesis Ninta, Crotalus horridus, Naja, Red Chinese, Siberian and Korean Ginseng, Goldenseal, Chamomile, and Barberry.

Each of the components can be used in the amount of about 0.5 to 5%, preferably, 0.5 to 2.0% by weight.

Prostoglandins have been found to improve nerve conduction velocity. However, it has been formed to be advantageous to induce prostaglandin production at the site affected by the disease rather than infusing the entire body. Consequently, the inclusion of 0.5 to 5% by weight of the composition such as primose oil, flax oil, and crocetin have been found to be beneficial.

The addition of L-Arginine and arginine containing peptides have been found to not only produce nitrous oxide at the site to increase blood circulation but also to attract hormones which can help the healing process. Additionally, L-arginine acts as a carrier for obtaining deeper penetration of some of the active ingredients when used in an amount of 0.5 to 5% by weight and can be generally used to produce the nitrous oxide.

Capsaicin has been recognized as aiding blood circulation and masking or relieving pain. Capsaisin can be used especially to relieve pain.

Since cracking and dryness of the feet are some of the symptoms of the diseases involved, namely, anhidrosis it has been found to be advantageous to include skin conditioning components such as graphite and trace minerals.

Because the body chemistry of individuals are different and the diseases may be in different phases it is preferable in some cases to use a variety of different components which can produce the desired effect on contact to produce a synergism.

The amounts of the different constituents of the compositions according to the invention are those traditionally used in the pharmaceutical field.

When the composition of the invention is an emulsion, the proportion of the fatty phase can range from 5% to 80% by weight and preferably from 5% to 50% by weight, relative to the total weight of the composition. The oils, emulsifiers and coemulsifiers used in the composition in emulsion form are chosen from those traditionally used in the cosmetics. The emulsifier and the coemulsifier are present in the composition in a proportion ranging from 0.3% to 30% by weight, and preferably 0.5 to 30% or, better still from 0.5 to 20%, by weight, relative to the total weight of the composition. The emulsion can, in addition, contain lipid vesicles.

When the compositions of the invention is an oily gel or solution, the fatty phase can represent more than 90% of the total weight of the composition.

In a known manner, the composition of the invention may also contain adjuvants which are customary in the pharmaceutical field, such as hydrophilic or lipophilic gelling agents, hydrophilic or lipophilic active agents, preservatives, antioxidants, solvents, perfumes, fillers. The amounts of these different adjuvants are those traditionally used in the pharmaceutical or dermatological field, and are, for example, from 0.01% to 10% of the total weight of the composition. Those adjuvants, depending on their nature, may be introduced into the fatty phase, into the aqueous phase and/or lipid spherules.

As oils which can be used in the invention, are those which do not clog or block pores such as vegetable oils, liquid fraction of sheat butter, sunflower oil and animal oils (perhydrosquantene) may be mentioned. Mineral oil lanolin and petroleum oils block pores so as to prevent the skin from releasing toxins.

Fatty alcohols and fatty acids (stearic acid) may also be used as fatty substances.

As emulsifiers which can be used in the invention, glycerol stearate, polysorbate 60 and the PEG-6/PEG-32/glycol stearate mixture sold under the name of Tefose® 63 by the company Gattefosse may be mentioned as examples.

As hydrophilic gelling agents, carboxyvinyl polymers (carbomer), acrylic copolymers such as acrylate/alkylacylate copolymers, polyacrylamides, polysaccarides such as hydroxypropylcellulose, clays and natural gums may be mentioned.

Lipophilic gelling agents, clays such as bentones, metal salts of fatty acids such as aluminum stearates and hydrophobic silica, or alternatively ethylcellulose and polyethylene may be mentioned.

Natural gums which may be used includes xantham gum, alginates and gelation. As hydrophilic active agents, proteins or protein hydrolysates, amino acids, polyols, urea, allantonin, sugars and sugar derivatives, water-soluble vitamins, starch and plant extracts, in particular those of the Aloe vera may be used.

As lipophilic active, agents, retinol (vitamin A) and its derivatives, tocopherol (vitamin E) and its derivatives, essential fatty acids, ceramides and essential oils may be used. These agents add extra moisturizing or skin softening features when utilized.

The compositions of the invention may include other plant or herbal extracts which reduce irritation. For example, there may be utilized extracts of Paraguay tea, Kola and Guarana, which provide a source of methylxanthines, saponius, tannins, and glycosides that have been shown to be anti-inflammatory and can be used to treat irritations and cracked skin.

Suitable herbs which can be used also to help healing of the skin include symphytum, officianalis, Moschus moscheferous, Cow bezoar, Pripalia geniculata, Plantago asiatica, Causticum, Helianthemum canadense, Ornithogalum umbellatum, Clematis crispa, Impatiens pallida, Pruaus Cerasus, and the like.

A surfactant can be included in the composition so as to provide deeper penetration of the ingredients. Although natural surfactants are preferred others such as isopropyl myristatc can be used.

Other ingredients may be used, preferably, in amounts from about 0.5% to 2% by weight.

The following examples illustrating the compositions of the invention are not intended to limit the scope of the invention. The amounts indicated are by weight percent unless otherwise noted.

### EXAMPLE 1

A homeopathic gel prepared by admixing the following ingredients.

| Ingredient | Wt. % |
|---|---|
| Carbomer 940 | 2.10 |
| Xantham Gum | 0.15 |
| Propylene glycol | 51.94 |
| Dipropylene glycol | 10.00 |
| Ethoxydiglycol | 15.00 |
| Dimethylisosorbide | 10.00 |
| Aloe Vera gel | 2.00 |
| Surfactant | 0.05 |
| Arnica Montana | 2.50 |
| Echinacea | 2.50 |
| Lachenis Malta | 2.00 |
| Crotalus horridus | 1.76 |
| | 100.00% |

Although the specific activity of each of either plants or herbs have been recognized, it has been surprisingly found that the combination as now claimed has been found to produce the desired effect.

### EXAMPLE 2

A crème was formed by admixing the following ingredients.

| | |
|---|---|
| Ingredient | Wt. % |

| | | |
|---|---|---|
| Aesculus hippocastanum extract | 6 | mcg/g |
| Arnica Montana extract | 6 | mcg/g |
| L-arginine | 6 | mcg/g |
| Echinacea angustfolia extract | 6 | mcg/g |
| Rhus toxicondendron extract | 6 | mcg/g |
| Ruta graveoleus extract | 6 | mcg/g |
| Graphites | 6 | mcg/g |
| Crotalus horridus extract | 0.08 | mcg/g |
| Heloderma horridum extract | 0.08 | mcg/g |
| Lachesis extract | 0.08 | mcg/g |
| Naja extract | 0.08 | mcg/g |
| Isopropyl myristate | 30 | mg/g |
| Brij 72 | 2 | mg/g |
| Brij 72IS | 2 | mg/g |
| Water | 83 | mg/g |
| Methyl paraben | 0.2 | mg/g |
| Propyl paraben | 20 | mcg/g |
| Glycerine | 3 | mg/g |
| 20% NaOH | 3 | mg/g |
| Dowcil 200 | 50 | mcg/g |
| Akodel 112 | 2 | mg/g |

The crème may be used to treat cracked skin on feet or anhidrosis.

### EXAMPLE 3

A lotion is prepared by admixing the following ingredients:

| Ingredient | Wt. % |
|---|---|
| Ginger | 1.10 |
| Propylene Glycol Stearate | 6.50 |
| Isocetyl alcohol | 5.00 |
| PEG-100 Stearate | 1.20 |
| Water | 69.90 |
| Echinacea | 3.00 |
| Methyl paraben | 0.20 |
| Propylene glycol | 12.00 |
| Sorbitan palmitate | 0.60 |
| Arnica Montana | 3.00 |
| Aesculus hippocastanum | 3.00 |
| Mate extract | 0.50 |
| | 100% |

If desired, 3% by weight capsaicin can be added. The lotion can be used to treat a patient suffering from fibromyalgia.

### EXAMPLE 4

Preparation of a Gel

| Ingredients | % W/W |
|---|---|
| Arnica Montana | 5.0 |
| Primrose oil | 3.0 |
| Arginine base (10% solution) | 5.0 |
| (Ajinomoto) | |
| Carbopol 940 | 0.4 |
| Butylene glycol | 6.5 |
| Echinacea | 3.0 |
| Chamomile glycolic extract | 3.0 |
| Crotalus horridus | 0.5 |
| Preservative | 0.1 |
| Fragrance | 0.1 |
| Deionized water | g.s. |
| | 100% |

To 20 ml of water with stirring is added the Carbopol 940. The mixture is stirred until hydration is complete and then butylene glycol is added. The arginine base is then added to the mixture. The remain ingredients are mixed together and added to the first mixture. The mixing is continued until uniform.

EXAMPLE 5

A gel is prepared by admixing the following ingredients.

| | Ingredient | Wt. % |
|---|---|---|
| 1. | Propylene Glycol | 43.00 |
| 2. | Polyacrylic acid | 2.10 |
| 3. | Dipropylene Glycol | 16.00 |
| 4. | Xantham Gum | 0.15 |
| 5. | Ethoxydiglycol | 15.00 |
| 6. | Dimethylisosorbide | 10.00 |
| 7. | Ascorbic Acid | 2.00 |
| 8. | Chloroxylenol | 0.20 |
| 9. | Linoleamidopropyl PG-diammonium | 1.50 |
| | chloride phosphate | |
| 10. | Glycereth 4.5 Lactate | 2.00 |
| 11. | Naja | 2.00 |
| 12. | Echinacea | 2.00 |
| 13. | Octoxynol - 9 | 0.50 |
| 14. | Primrose Oil | 2.00 |
| 15. | Cocamidopropyl PG-dimon | 1.00 |
| | chloride phosphate | |
| 16. | Water | 6.00 |
| 17. | Ginger | 0.44 |

Ingredients 1 and 2 are mixed to disperse and form a gel. About 80% of ingredient 3 is mixed with ingredient 4, added to the gel slightly heated with admixture. The balance of 3 is mixed with ingredients 5-17 and added to the gel at 38 degrees C. After mixing, the pH is adjusted to about 4 and then the gel is brought to room temperature.

Arnica Montana may also be added to have a plurality of dual acting ingredients. Capsiacin together with the ginger can be used to relieve pain.

What is claimed is:

1. A method for treatment of a disease in a patient wherein there is restricted blood flow and a reduction in motor and sensory nerve conduction velocities which comprises topically administering to said patient an effective amount of a composition comprising:
A. About 0.5 to 5% by weight of a vasodilator derived from a plant;
B. About 0.5 to 5% by weight of a stimulator of lymphatic activity derived from a plant;
C. About 0.5 to 5% by weight of a mobilizer of white blood cell activity derived from a plant and the remainder being filler.

2. The method of claim 1 wherein said vasodilator is selected from the group consisting of Arnica Montana, primrose oil, rosemary, ginger, Hamamelis, yohimbe, and niacin L-ornithate or a mixture thereof.

3. The method of claim 1 wherein said stimulator of lymphatic activity comprises Echinacea Augustifolia.

4. The method of claim 1 wherein said mobilizer of white blood cell activity is selected from the group consisting of Lachesis ninta, Crotalus horridus and Naja.

5. The method of claim 1 wherein said composition includes L-arginine.

6. The method of claim 1 wherein a plant extract which includes the production of prostaglandin activity at the site of application.

7. The method of claim 1 wherein said plant extract is selected from the group consisting of primrose oil, flaxseed oil and crocitin.

8. The method of claim 1 wherein said composition contains skin conditioning components.

9. The method of claim 8 wherein said skin conditioning components comprise graphite and trace minerals.

10. The method of claim 1 wherein said disease is fibromyalgia.

11. The method of claim 1 wherein said disease is diabetic neuropathy.

12. The method of claim 1 wherein said composition is a gel, cream, emulsion, salve or lotion.

13. The method of claim 1 including Rhus hippocastanum and Aesculus hippocastanum.

14. The method of claim 1 wherein said composition includes a pain relieving amount of capsaicin.

15. A method for the treatment of a disease in a patient wherein there is a restriction of blood flow and motor and sensory nerve conduction velocities which comprises topically administering a composition comprising:
A. About 0.5 to 5% by weight of Arnica Montana;
B. About 0.5 to 5% by weight of Echinacea Augustofolia;
C. About 035 to 5% by weight of a plant extract selected from the group consisting of Lachesis ninta, Crotalus horridus, Naja and mixtures thereof, and the remainder being fillers.

16. The method of claim 14 wherein said composition includes L-arginine.

17. The method of claim 14 wherein said composition includes ginger.

18. The method of claim 14 wherein said composition includes at least one member selected from the group consisting of Rhus toxicodendron, Rhus graveoleus, Resculus hippocastanum and Prunus Cerasus.

19. The method of claim 14 wherein said composition contains graphite and trace minerals.
